(19) United States
(12) Patent Application Publication
(10) Pub. No.: US 2025/0195739 A1
(43) Pub. Date: Jun. 19, 2025

(54) Devices And Methods For Estimating A Blood Component In Fluid Within A Canister Of A Medical Waste Collection System

(71) Applicant: Stryker Corporation, Portage, MI (US)

(72) Inventors: Chamara L. Gamhewage, Kalamazoo, MI (US); Siddarth Satish, Portola Valley, CA (US); Steven D. Scherf, Oakland, CA (US); Kevin J. Miller, Mountain View, CA (US); Benjamin Edinger, Grand Haven, MI (US); Jeffrey Conway, Incline Village, NV (US); Russell Conway, Capitola, CA (US)

(73) Assignee: Stryker Corporation, Portage, MI (US)

(21) Appl. No.: 18/847,856

(22) PCT Filed: Mar. 17, 2023
(86) PCT No.: PCT/US2023/015437

§ 371 Date: Sep. 17, 2024

Related U.S. Application Data
(60) Provisional application No. 63/321,415, filed on Mar. 18, 2022.

(51) Int. Cl. A61M 1/00

(52) U.S. Cl. CPC A61M 1/60; A61M 2205/3306; A61M 2209/10

(57) ABSTRACT

An insert and insert assembly for facilitating image-based determination of a blood component in medical waste. The medical waste is collected in a waste canister of a medical waste collection system that includes a vacuum source, a fluid measuring subsystem, and a cleaning subsystem. The insert assembly includes means for positioning the insert within the waste canister for an imaging feature of the insert to be spaced apart from an inner surface of the waste canister. The imaging feature may include imaging surfaces in a side-by-side arrangement with each being spaced apart from the inner surface by a respective distance. The insert may include a flow surface configured to limit obstruction of pressurized fluid from the cleaning subsystem being directed at the imaging feature. The insert may define a slot providing fluid communication between a rear side and a front side of the insert.

### PRIORITY CLAIM

This application claims priority to and all the benefits of U.S. Provisional Patent Application No. 63/321,415, filed on Mar. 18, 2022, the entire contents of which are hereby incorporated by reference.

### BACKGROUND

A byproduct of surgical procedures is the generation of liquid, semisolid, and/or solid waste material. The medical waste may be removed from the surgical site through a suction tube under the influence of a vacuum provided by a medical waste collection system. The medical waste may be collected within a waste canister of the medical waste collection system and include liquids such as blood, interstitial fluid, mucus, irrigating fluid, and the like.

Determining blood loss during surgery may be used to monitor intraoperative patient health. Advances in imaging and computing have provided for quantifying blood loss by capturing an image of the fluid-containing media, such as a canister. One such system is sold under the tradename Triton by Gauss Surgical, Inc. (Menlo Park, Calif.) and disclosed in commonly-owned U.S. Pat. No. 9,773,320, issued Sep. 26, 2017, the entire contents of which are hereby incorporated by reference. The system includes an insert within a freestanding canister with the insert configured to permit a thin layer of fluid to be situated between the insert and an inner surface of the canister. The use of a freestanding canister consumes valuable space within the operating suite, and the freestanding canister fails to leverage certain features and subsystems integrated into the waste canister of the medical waste collection system. Therefore, there is a need in the art to address the aformentioned technical challenges.

### SUMMARY

The present disclosure is directed to devices and methods for quantifying a blood component in medical waste disposed within a waste canister of a medical waste collection system. The waste canister defines a waste volume for receiving and collecting the waste material. A vacuum source may be supported on the chassis and configured to draw suction on the waste canister through one or more internal lines. A front casing defines at least one cutout or window to facilitate capturing an image of the waste canister with an optical sensor, e.g., on a device such as a smartphone or tablet. The front casing may include a lip or portion that obscures a lower portion of the waste canister. The medical waste collection system includes a fluid measuring subsystem and a cleaning subsystem. The fluid measuring system is configured to measure a fluid level of the waste material disposed within the waste canister. Based on signals received from the fluid measuring subsystem, the controller is configured to determine a fluid volume of the waste material in the waste canister. The controller may determine a volume of blood within the waste canister based on the determined fluid volume and the blood component determined by the controller through the image-based processing. The cleaning subsystem includes sprayers rotatably disposed within the waste canister and configured to direct pressurized liquid against an inner surface of the waste canister. A canister light may be configured to illuminate an interior of the waste canister.

An insert is disposed within the waste canister. The insert includes several geometries, at least one of which is an imaging feature configured to be spaced apart from an inner surface of the waste canister to define a gap of known and fixed distance. The gap permits a thin layer of fluid to be situated between the insert and the inner surface of the waste canister. The insert generally includes a front side opposite a rear side. The insert may include upper, lower, and lateral sides extending between the front side and the rear side.

The imaging feature includes a first imaging surface, and optionally a second imaging surface. The first imaging surface is spaced apart from the inner surface by a first distance and the second imaging surface is spaced apart from the inner surface by a second distance. The second distance may be greater than the first distance. The first imaging surface and the second imaging surface may be positioned lateral to one another in a side-by-side arrangement. The first imaging surface and the second imaging surface separated by a ridge having a thickness equal to a difference between the first distance and the second distance. The imaging feature may be a square or rectangular, and each of the first and second imaging surfaces may be square or rectangular. Alternatively, the insert may include three, four, or five or more imaging surfaces. In another variant, the imaging feature is formed of singular surface that has a tapered profile between the lateral sides of the insert. A thickness of a first of the lateral sides is greater than a thickness of a second of the lateral sides.

A lower aspect may extend upwardly from the lower side and include a front surface configured to be positioned adjacent to or in an abutting relationship with the inner surface of the waste canister. The first imaging surface and the second imaging surface may be recessed from the front surface of the lower aspect. An upper aspect may extend downwardly from the upper side and includes a front surface configured to be positioned adjacent to or in an abutting relationship with the inner surface of the waste canister. The front surface of the upper aspect may be shaped the same as the front surface of the lower aspect, for example, contoured to a curvature of the inner surface. The imaging feature may be recessed from the front surface of the upper aspect. The imaging feature may be positioned between the lower aspect and the upper aspect. The upper aspect may be sized to be at least equal to a reference marker configured to be detected by the optical sensor when capturing the image of the waste canister. A height of the lower aspect may be sized to position the imaging feature above the lip and within the window.

The lateral arrangement of the first imaging surface and the second imaging surface may be based on a direction of rotation of the sprayers of the cleaning subsystem. The first and second imaging surfaces may be arranged so that the pressurized liquid contacts the first imaging surface prior to contacting the second imaging surface. In certain implementations, the insert may include a flow surface configured to direct the pressurized liquid from the cleaning subsystem towards the imaging surface. The flow surface may be associated with the upper aspect and slope downwardly and radially outwardly. Additional geometries may be associated with the flow surface, for example, channels, lumens, and undulations.

In certain implementations, the insert may define a slot providing fluid communication from the rear side to the front side. The slot may be positioned widthwise along the insert and located above the imaging feature. The upper aspect may define a channel extending between the upper side of the insert and the slot.

The insert may be provided on an insert assembly. The insert assembly includes means for supporting the insert within the waste canister to locate and maintain a position the imaging feature. The means for supporting the insert may couple the insert assembly to the upper cap of the waste canister, the lower portion of the waste canister, a sidewall of the waste canister, or combinations thereof. Implementations of the insert and implementations of the means for supporting the insert within the waste canister are interchangeable.

The insert assembly may include a mounting head, a strut, and the insert. The mounting head is configured to be secured to the upper cap. The strut may be a resiliently flexible plate, or a pair of resiliently flexible rods. The strut may be biased such that, with the mounting head secured to the upper cap, the insert is urged into direct contact with the inner surface. The rods are coupled to the lateral sides of the insert to provide a fulcrum to permit the insert to pivot to maintain optimal contact between the insert and the inner surface. The front side of the insert may include feet. A depth of the feet may dictate the gap between the imaging feature and the inner surface. The feet may be in a triangular arrangement.

In certain implementations, the insert assembly includes a coupler housing, at least one magnet, and the insert. The coupler housing may be contoured to an outer surface and the lower portion of the waste canister. The coupler housing may be aligned with at least one rib of the waste canister. An upper member of the coupler housing may be positioned lateral to the insert. The insert may include a dimple or post to directly contact the inner surface and further support the insert in the desired position.

In certain implementations, the insert assembly is secured to the sensor rod of the fluid measuring subsystem. The insert assembly may include a flange extending from the insert and coupled to a mounting hub defining an opening sized greater than the sensor rod. A locking member may secure the mounting hub to the sensor rod. The insert is prevented from rotation and thereby locked in the desired position.

In certain implementations, the insert assembly includes a frame. The frame may include a lower support ring, an upper support ring, and braces fixedly securing the upper support ring to the lower support ring. The insert is fixedly coupled to the frame. The braces may have a height defined between the lower support ring and the upper support ring to approximate a height of the waste canister. In one variant, the lower support ring includes a front feature having a height sized to position the imaging feature above the lip and within the window. The front feature may also include a mount configured to secure the insert to the frame. In another variant, resilient tongues extend upwardly from the lower support ring. The tongues are dimensioned and oriented relative to the lower support ring to be resiliently deflected inwardly as the frame is deposited into the waste canister. In still another variant, the lower support ring defines apertures configured to threadably receive set screws.

In certain implementations, the insert assembly includes a turnbuckle securing the insert within the waste canister. The turnbuckle movably couples a front mount to a rear mount. The front mount may include a front surface contoured to the inner surface of the waste canister. The imaging feature may be positioned lateral to the front mount, but other suitable arrangements are contemplated.

In certain implementations, adhesive is used to affix the insert within the waste canister. The insert assembly includes at least one leg coupled to or integrally formed with the insert. The legs are configured to be affixed to the waste canister to position the first imaging surface at the first distance from the inner surface. The legs may be pairs of legs are spaced apart from each other vertically along a body.

Therefore, according to first aspect of the present disclosure, the insert assembly includes an insert, and means for positioning the insert within the waste canister. The insert comprises an imaging feature configured to be imaged with an optical sensor for an image be processed for quantifying the blood component. The imaging feature comprises a first imaging surface and a second imaging surface. The means for positioning the insert within the waste canister causes a front side of the insert to engage an inner surface of the waste canister. The means further provide for spacing apart the first imaging surface from the inner surface by a first distance, and spacing apart the second imaging surface from the inner surface by a second distance greater than the first distance. The first imaging surface and the second imaging surface are positioned lateral to one another in a side-by-side arrangement.

The first distance may be within a range of 1.2 to 3.7 millimeters, and wherein the second distance is within a range of 1.7 to 4.2 millimeters. The first imaging surface and the second imaging surface may be separated by a ridge having a thickness equal to a difference between the first distance and the second distance. The thickness of the ridge may be approximately 0.5 millimeters.

The insert may include at least three feet extending forward of the imaging feature and configured to directly contact the inner surface of the waste canister. The feet may be in a triangular arrangement so as to allow for wobble and vertical self-centering of the insert against the inner surface of the waste canister. The insert defines at least one cavity extending inwardly from a rear side opposite the front side.

The means for positioning the insert within the waste canister may include a mounting head configured to be coupled to an upper cap of the waste canister, and a strut coupling the mounting head with the insert, wherein the strut is biased or formed to urge the insert into direct contact with the inner surface of the waste canister. The strut may be a plate, or a pair of rods. The rods may be coupled to one another with a crossmember in a U-shaped arrangement. The insert may define at least one slot within which the strut is pivotably disposed so as to permit the insert to pivot about the strut. Alternatively, the means for positioning the insert within the waste canister may include at least one of a magnet, a locking member, a frame, tongues, a turnbuckle, and adhesive.

In certain implementations, the insert includes a lower aspect defined between a lower side of the insert and the imaging feature. The lower aspect is sized relative to a lip of the medical waste collection system to position the imaging feature within a window of the medical waste collection system through which the waste canister is visible. The insert may include an upper aspect defined between an upper side of the insert and the imaging feature. The imaging feature may be recessed from the upper aspect.

The first imaging surface may be positioned relative to the second imaging surface based on a direction of the rotatable sprayer so as to encounter the pressurized liquid prior to the second imaging surface. The insert may include a flow surface configured to direct the pressurized liquid from the cleaning subsystem towards the imaging feature. The flow surface may be sloped and oriented to be aligned with an incoming direction of the pressurized liquid. A slot may be located above the imaging feature and configured to provide fluid communication from a rear side to the front side of the insert. The upper aspect may define a channel extending between the upper side of the insert and the slot.

According to a second aspect of the present disclosure, the insert assembly includes a mounting head configured to be coupled to the upper cap of the waste canister, and a strut coupled to the mounting head. The insert is coupled to the strut and comprising an imaging feature configured to be imaged with an optical sensor for an image be processed for quantifying the blood component. The strut is biased or formed to urge the insert into direct contact with an inner surface of the waste canister for the imaging feature to be spaced apart from the inner surface.

According to a third aspect of the present disclosure, the insert assembly includes an insert, and the means for positioning the insert within the waste canister to cause a front side of the insert to engage an inner surface of the waste canister, and to space apart the imaging feature from the inner surface. The insert includes an imaging feature configured to be imaged with an optical sensor for an image be processed for quantifying the blood component. The imaging feature has a tapered profile extending between opposing lateral sides of the insert.

### BRIEF DESCRIPTION OF THE DRAWINGS

FIG. 1 shows a medical waste collection system configured to removably receive a manifold. The medical waste collection system draws medical waste through a suction tube and the manifold to be collected in a waste canister.

FIG. 2 shows the medical waste collection system of FIG. 1 with a front casing removed. An optical sensor is configured to capture an image of the waste canister and an insert disposed within the waste canister. The optical sensor may be on a mobile device.

FIG. 3 is a perspective view of an implementation of the insert.

FIG. 4 is a perspective view of another implementation of the insert.

FIG. 5A is a perspective view of an implementation of an insert assembly that includes the insert of FIG. 3.

FIG. 5B is a perspective view of the insert assembly of FIG. 5A disposed within the waste canister.

FIG. 6A is a perspective view of another implementation of the insert assembly disposed within the waste canister.

FIG. 6B is a rear perspective view of the insert assembly of FIG. 6A.

FIG. 6C is a top perspective view of a variant of the insert of FIG. 6A.

FIG. 7A is a perspective view of another implementation of the insert assembly disposed within the waste canister.

FIG. 7B is a perspective view of the insert assembly of FIG. 7A disposed within the waste canister.

FIG. 8A is a perspective view of another implementation of the insert assembly disposed within the waste canister.

FIG. 8B is a bottom plan view of the arrangement of FIG. 8A in which a coupler housing of the insert assembly is supported against a rib of the waste canister.

FIG. 8C is a perspective view of the arrangement of FIG. 8A. A reference marker is affixed to the waste canister.

FIG. 9A is an exploded perspective view of another implementation of the insert assembly that includes the insert of FIG. 4.

FIG. 9B is a perspective view of the insert assembly of FIG. 9A disposed within the waste canister and with the reference marker affixed to the waste canister.

FIG. 10A is a perspective view of another implementation of the insert assembly.

FIG. 10B is a perspective view of the insert assembly of FIG. 10A disposed within the waste canister.

FIG. 11 is a perspective view of another implementation of the insert assembly.

FIG. 12 is a perspective view of another implementation of the insert assembly.

FIG. 13 is a perspective view of another implementation of the insert assembly.

FIG. 14 is a perspective view of a rear side of the insert of FIGS. 11-13 in which a slot provides fluid communication to an imaging feature of the insert.

FIG. 15A is a perspective view of another implementation of the insert assembly.

FIG. 15B is a perspective view of the insert assembly of FIG. 15A disposed within the waste canister.

FIG. 16A is a perspective view of another implementation of the insert assembly.

FIG. 16B is a perspective view of the insert assembly of FIG. 16A disposed within the waste canister.

### DETAILED DESCRIPTION

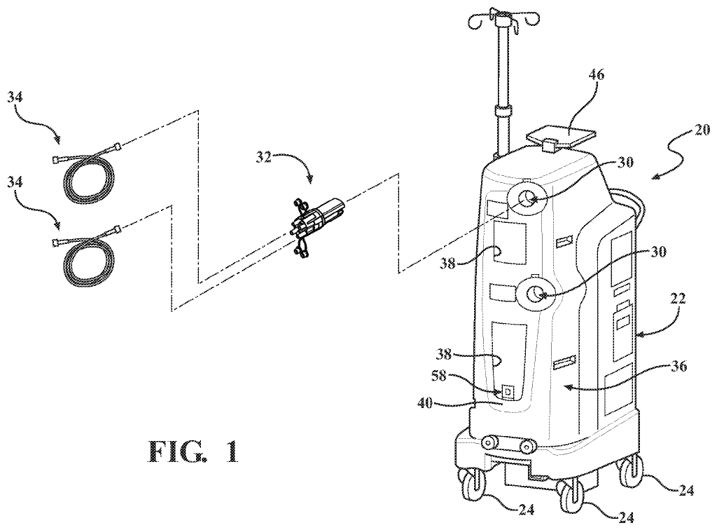

FIGS. 1 and 2 show a medical waste collection system 20 for collecting waste material generated during medical procedures. The waste material may include semisolid and solid matter such as tissue, and liquid matter such as blood mixed with irrigating liquids and other bodily fluids. The medical waste collection system 20 collects and stores the waste material until it is necessary or desired to empty and dispose of the waste material with a docking station. An exemplary docking station is disclosed in commonly-owned U.S. Pat. No. 7,401,898, issued Nov. 24, 2009, the entire contents of which are hereby incorporated by reference.

The medical waste collection system 20 may include a chassis 22 and wheels 24 for moving the chassis 22 along a floor surface within a medical facility. The medical waste collection system 20 includes at least one waste canister 26 defining a waste volume for receiving and collecting the waste material. A vacuum source 28 may be supported on the chassis 22 and configured to draw suction on the waste canister 26 through one or more internal lines. The vacuum source 28 may include a vacuum pump and a vacuum regulator supported on the chassis 22 and in fluid communication with the waste canister 26. The vacuum regulator is configured to regulate a level of the suction drawn by the vacuum pump on the waste canister 26. Suitable construction and operation of several subsystems of the medical waste collection system 20 are disclosed in commonly-owned U.S. Pat. No. 7,621,898, issued Nov. 24, 2009, U.S. Pat. No. 10,105,470, issued Oct. 23, 2018, and U.S. Pat. No. 11,160,909, issued Nov. 2, 2021, the entire contents of which are hereby incorporated by reference.

The medical waste collection system 20 includes at least one receiver 30 supported on the chassis 22. The receiver 30 defines an opening sized to removably receive at least a portion of a manifold 32. A suction path may be established from suction tubes 34 to the waste canister 26 through the manifold 32 removably inserted into the receiver 30. In other words, the vacuum generated by the vacuum source 28 is drawn on the suction tubes 34, and the waste material at the surgical site is drawn through the manifold 32, through the receiver 30, and into the waste canister 26. The manifold 32 may be a disposable component. Exemplary implementations of the receiver 30 and the manifold 32 are disclosed in commonly-owned U.S. Pat. No. 10,471,188, issued Nov. 12, 2019, the entire contents of which are hereby incorporated by reference.

The chassis 22 includes a front casing 36 that defines at least one cutout or window 38 to facilitate capturing an image of the waste canister 26 with an optical sensor 88. As schematically represented in FIG. 2, the field of view of the optical sensor 88 includes the waste material collected in the waste canister 26. The optical sensor 88 may be on a mobile device 89. Optical properties of the waste material may be analyzed and processed to quantify a blood component in the waste material. Exemplary methods for quantifying the blood component are disclosed in commonly-owned U.S. Pat. No. 8,792,693, issued Jul. 29, 2014, the entire contents of which are hereby incorporated by reference. The waste canister 26 may be formed from a transparent or optically clear material. The front casing 36 may include a lip or portion (generally identified at 40) that obscures a lower portion 54 of the waste canister 26.

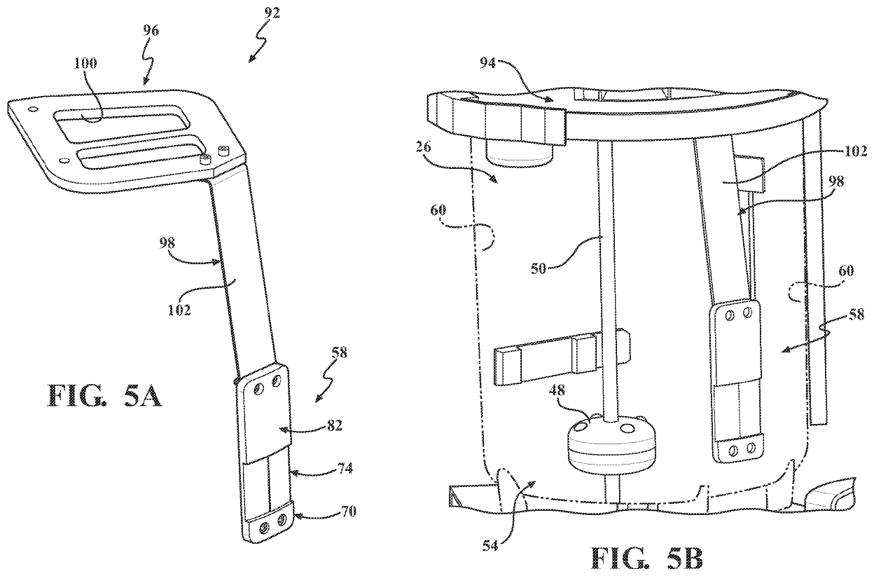

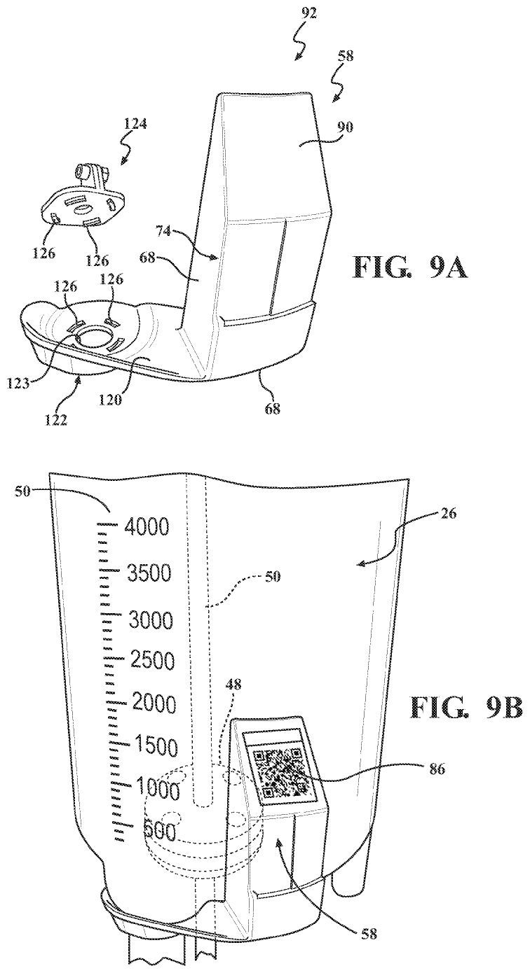

With continued reference to FIG. 2, the medical waste collection system 20 includes a fluid measuring subsystem 42 configured to measure a fluid level of the waste material disposed within the waste canister 26. The fluid measuring subsystem 42 is in communication with a controller 44 disposed on the chassis 22 (or another processor), or alternatively processing of data may be performed remotely. An exemplary implementation of the fluid measuring subsystem 42 is disclosed in the aforementioned U.S. Pat. No. 7,621,898 in which a float element 48 is movably disposed along a sensor rod 50 (see FIGS. 5B and 9B). Based on signals received from the fluid measuring subsystem 42, the controller 44 is configured to determine a fluid volume of the waste material in the waste canister 26. The controller 44 may determine a volume of blood within the waste canister 26 based on the determined fluid volume and the blood component determined by the controller 44 through the image-based processing. The volume of blood within the waste canister 26 may be indicative of the blood loss of the patient. The blood loss may be displayed on a user interface 46 disposed on the chassis 22, and/or on another user interface such as the mobile device 89 or another display. Alerts may be provided via the user interface 46 should the blood loss of the patient exceed predetermined or selected limits. The blood loss data associated with the medical procedure may be transmitted to an electronic medical record.

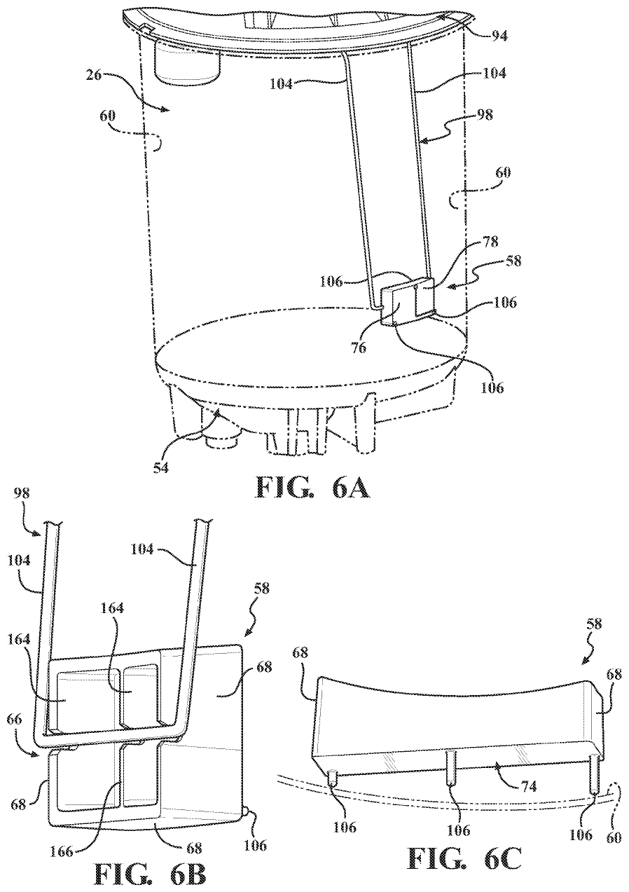

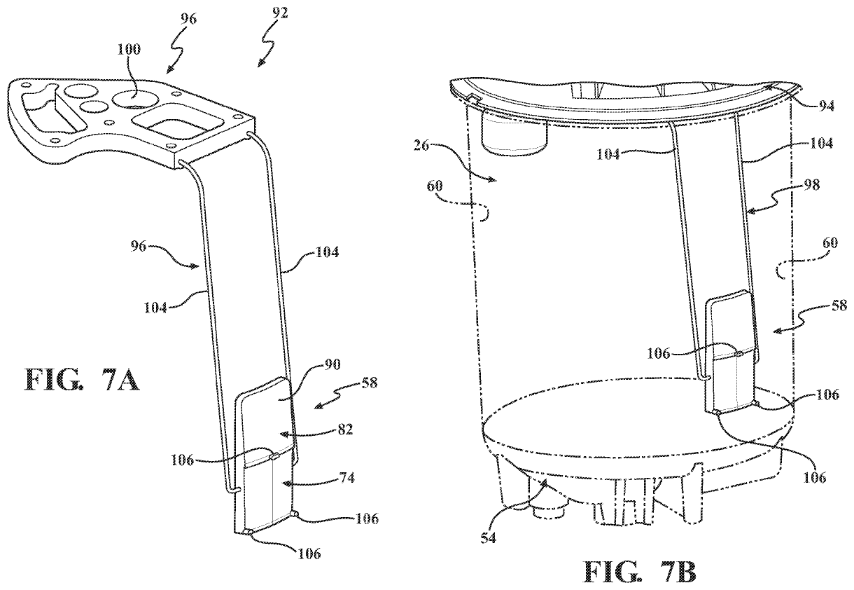

The medical waste collection system 20 includes a cleaning subsystem 52. An exemplary implementation of the cleaning subsystem 52 is disclosed in the aforementioned U.S. Pat. No. 10,105,470 in which sprayers are rotatably disposed within the waste canister 26 and configured to direct pressurized liquid against an inner surface 60 of the waste canister 26. The cleaning subsystem 52 may be activated based on an input to the user interface 46, or upon docking the medical waste collection system 20 with the docking station. The docking station may provide water and detergent to be directed through the sprayers to clean the waste canister 26, and empty the waste canister 26 during or upon completion of the cleaning cycle. Thereafter, a prefill pump (not identified) in fluid communication with a liquid reservoir supported on the chassis 22 may be operated to direct a prefill liquid into a lower portion 54 of the waste canister 26. The prefill liquid is at a level above a frustoconical-like shape of the lower portion 54 (see FIGS. 6A and 7B), and the float element 48 of the fluid measuring subsystem 42 rises correspondingly. The controller 44 tares or sets a zero point of the fluid measuring subsystem 42, above which the geometry of the waste canister 26 more closely approximates a cylinder for volume determinations of the waste material. Further disclosure regarding operation of the prefill pump and the fluid measuring subsystem 42 is provided in the aforementioned U.S. Pat. No. 10,105,470.

The medical waste collection system 20 may also include a canister light 56 configured to illuminate an interior of the waste canister 26. Any suitable positioning of the canister light 56 within the waste canister 26 is contemplated with FIG. 2 being a non-limiting example. The canister light 56 may activated based on an input to the user interface 46, or a device in communication with the controller 44. In addition to improving visibility to the operator, the illumination of the waste canister 26 by the canister light 56 may lighten a tint or hue (or other optical property) of the waste contents for improved image-based processing. For example, an intensity of the light emitted from the canister light 56 may be known and accounted for in the image-based determinations of the blood component in the waste material.

It is known that fluids with higher concentrations of hemoglobin—either as free hemoglobin or intracellular hemoglobin—have a deeper red tint. Certain implementations of image-based determinations of the blood component extract a redness or other color component value to estimate the blood component (e.g., hemoglobin) within the blood, and more generally the blood component in the waste material. The deeper red tint may result in color signal saturation that itself may yield suboptimal readings and determinations. Likewise, overly opaque fluids may result from the liquid containing excessive red blood cells with insufficient free hemoglobin, or from lysis of an unknown portion of the whole red blood cells. To ensure consistent and accurate image-based determinations of the blood component, an insert 58 is configured to be disposed within the waste canister 26. The insert 58 includes several geometries, at least one of which is spaced apart from an inner surface 60 of the waste canister 26 to define a gap of known and fixed distance. The gap permits a thin layer of fluid to be situated between the insert 58 and the inner surface 60 of the waste canister 26. Further, the insert 58 may be white or nearly white. With a white backdrop to the thin layer of fluid, the fluid exhibits a region of at least substantially uniform color that is below a color intensity to cause signal saturation. Unlike a freestanding canister that is disposable, the insert 58 and insert assembly 62 includes features to permit the insert 58 to be integrated with the waste canister 26 of the medical waste collection system 20, which itself is a capital component. In other words, the interior of the waste canister 26 is designed not to be readily accessible by non-trained personnel. As a result, the insert 58 is configured to be reusable over multiple procedures in which the insert 58—which may be formed from a white plastic—may be repeatedly exposed to stain-inducing blood. Deviations in the original color of the insert 58 may subsequently result in inaccurate determinations, and the insert 58 and insert assembly 62 account for such considerations. It is further noted that the integration of the insert 58 with the waste canister 26 provides for real-time updating of blood loss, and improves sustainability by reducing environmental waste.

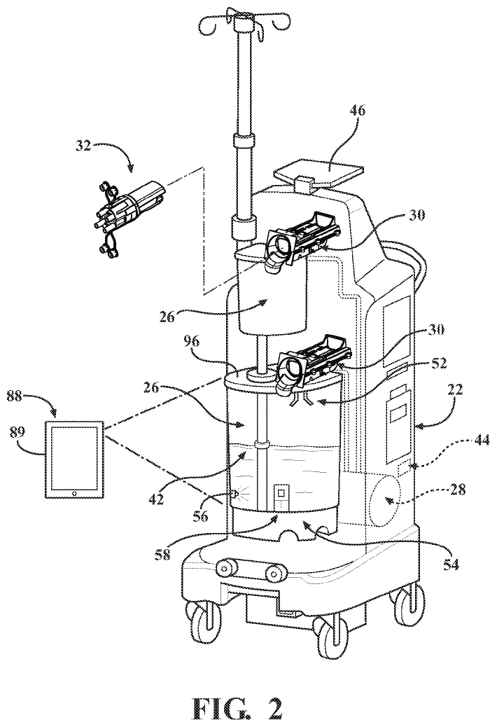

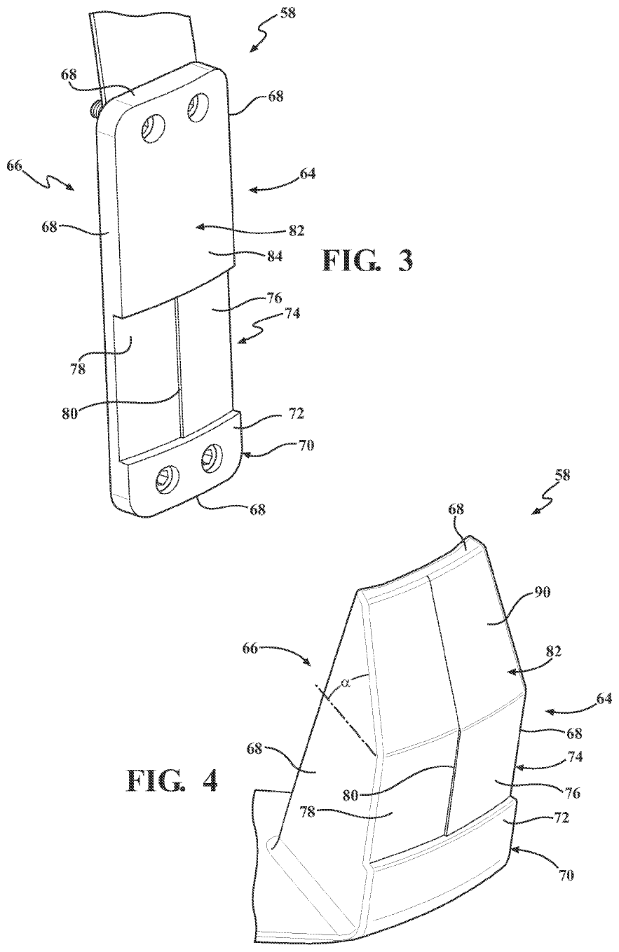

Referring to FIGS. 3 and 4, implementations of the insert 58 are shown with additional implementations to be later described. The insert 58 generally includes a front side 64 opposite a rear side 66. For convention, the front side 64 is configured to be positioned adjacent the inner surface 60 of the waste canister 26, and the rear side 66 is opposite the front side 64. The insert 58 may include upper, lower, and lateral sides 68 extending between the front side 64 and the rear side 66. A lower aspect 70 may extend upwardly from the lower side 68 and include a front surface 72 configured to be positioned adjacent to or in an abutting relationship with the inner surface 60 of the waste canister 26. An exemplary implementation includes the front surface 72 directly contacting the inner surface 60.

The insert 58 further includes an imaging feature 74. The imaging feature 74 includes a first imaging surface 76, and optionally a second imaging surface 78. The first imaging surface 76 and the second imaging surface 78 are recessed from the front surface 72 of the lower aspect 70. With the front surface 72 directly contacting the inner surface 60 of the waste canister 26, the first imaging surface 76 is spaced apart from the inner surface 60 by a first distance and the second imaging surface 78 is spaced apart from the inner surface 60 by a second distance. The second distance may be greater than the first distance. In one example, the first distance is 1.7 millimeters, and the second distance is 2.2 millimeters. It is more broadly contemplated that the first distance may be within the range of approximately 0.7 to 5.7 millimeters, and more particularly within the range of 1.2 to 3.7 millimeters, and the second distance may be within the range of approximately 1.2 to 6.2 millimeters, and more particularly within the range of 1.7 to 4.2 millimeters. The first distance and the second distance permit a thin layer of fluid to be situated between the first imaging surface 76 and the inner surface 60 and the second imaging surface 78 and the inner surface 60. The thin layer of fluid exhibits at least substantially uniform color, and the controller 44 is configured to locate the region of the image associated with the imaging feature 74 for the image-based determinations of the blood component. The second imaging surface 78, while optional (see FIGS. 6C and 11-16B), being a predefined geometry at the second distance provides a gradient of increasing color intensity being below the color intensity that may cause the signal saturation. The color gradient may be used by the controller 44 to improve the image-based determinations of the blood component, and further perform additional functionality such as those disclosed in the aforementioned U.S. Pat. No. 9,773,320. It should be appreciated that the insert 58 may include two, three, four, or five or more imaging surfaces with the illustrated implementations being non-limiting examples.

The first imaging surface 76 and the second imaging surface 78 may be positioned lateral to one another in a side-by-side arrangement. As opposed to a vertical arrangement akin to a staircase, the side-by-side arrangement, among other advantages, provides for the color gradient at lower fluid levels and improves cleanability of the insert 58. FIGS. 3 and 4 show the first imaging surface 76 and the second imaging surface 78 separated by a ridge 80 having a thickness equal to a difference between the first distance and the second distance. For example, the ridge 80 may be approximately 0.5 millimeters. It is contemplated that the ridge 80 may be radiused or otherwise contoured for aesthetics, manufacturability, or function. Other geometries for transitioning between the first imaging surface 76 and the second imaging surface 78 are contemplated.

The implementation of the insert 58 of FIG. 3 includes an upper aspect 82 extending downwardly from the upper side 68 and includes a front surface 84 configured to be positioned adjacent to or in an abutting relationship with the inner surface 60 of the waste canister 26. The front surface 84 of the upper aspect 82 may be shaped the same as the front surface 72 of the lower aspect 70. The front surfaces 72, 84 may be contoured to an inner diameter or the inner surface 60 to promote consistent positioning of the imaging feature 74 within the waste canister 26 and limit inadvertent movement of the insert 58 relative to the waste canister 26. The imaging feature 74 may be recessed from the front surface 84 of the upper aspect 82. The imaging feature 74 may be positioned between the lower aspect 70 and the upper aspect 82. In such an arrangement, the imaging feature 74 may be a square or rectangular.

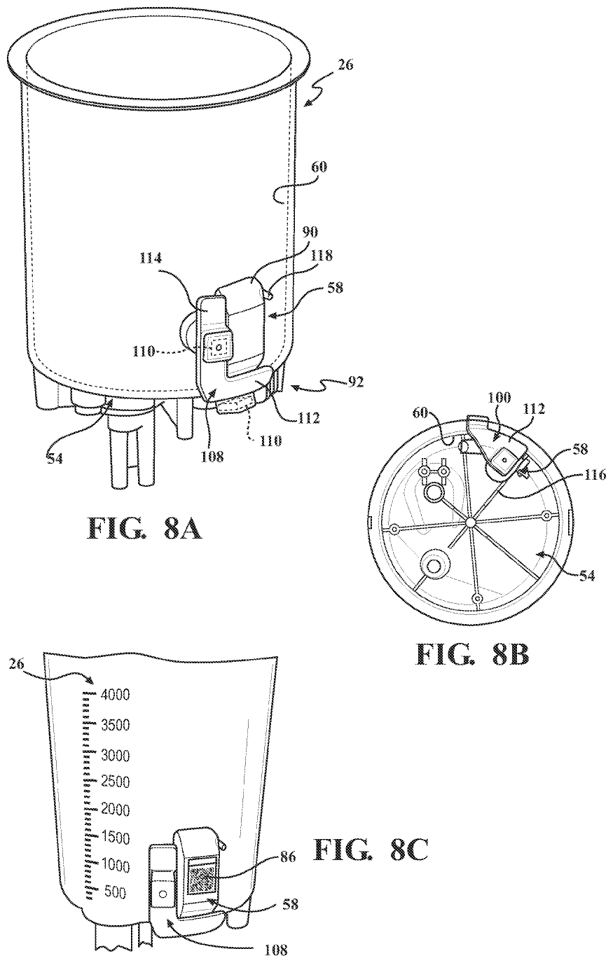

The upper aspect 82 may be sized to be at least equal to a reference marker 86 (see FIGS. 8C and 9B) configured to be detected by the optical sensor 88 when capturing the image of the waste canister 26. The reference marker 86 facilitates the controller 44 locating the region of the image associated with the imaging feature 74, and further provides for color-correction to compensate for variances in ambient lighting or other optical aberrations. An exemplary implementation of the reference marker 86 is disclosed in commonly-owned U.S. Pat. No. 9,824,441, issued Nov. 21, 2017, the entire contents of which are hereby incorporated by reference, in which a quick response (QR) code is affixed with adhesive to an outer surface of the waste canister 26 corresponding to a position of the upper aspect 82 of the insert 58 disposed within the waste canister 26. The QR code may be printed to be red of a known red color component value. The controller 44 may adjust values based on the red color component value as detected in the captured image relative to the known red color component value. Further, having the upper aspect 82 positioned behind and being equal to or larger in size than the reference marker 86 provides for improved image segmentation of the red QR code within the image that otherwise may include dark red tinted fluid in the waste canister 26. It is contemplated that the reference marker 86 may be affixed to the insert 58, in particular the upper aspect 82 of the insert 58. The reference marker 86 may include an adhesive backing, and an adhesive film may be disposed over the reference marker 86 and at least a portion of the insert 58. The adhesive film may cover the upper aspect 82, the imaging feature 74, and other geometries of the insert 58. The adhesive film may provide a hydrophobic coating to help prevent staining of the insert 58.

With continued reference to FIG. 3, the insert 58 is rectangular in shape with a height greater than a width. A thickness of the insert 58 defined between the front side 64 and the rear side 66 is relatively small such that the insert 58 is plate-like in construction. The shape maximizes a surface area of the front side 64, including the imaging feature 74, for a given volume of the insert 58. With the insert 58 disposed within the waste canister 26, it consumes a subvolume of the waste volume of the waste canister 26. It is desirable to minimize the consumed subvolume so as to limit its effect on the fluid measuring subsystem 42, particularly in implementations in which the insert 58 is retrofitted on an existing medical waste collection system. Alternatively, insert volumes of inserts of various shapes and sizes may be stored in a database and selected on the user interface 46 when installed. Based on the insert volume and the fluid level as measured by the fluid measuring subsystem 42, the controller 44 may be configured to reconcile the consumed subvolume and calculate the fluid volume with improved accuracy.

Referring now to FIG. 4, the insert 58 may include a flow surface 90 configured to direct the pressurized liquid from the cleaning subsystem 52 towards the imaging feature 74, or at least limit obstruction of the pressurized liquid being directed from the cleaning subsystem 52 towards the inner surface 60 of the waste canister 26. The sprayers of the cleaning subsystem 52 are rotatably coupled to an upper cap 94 of the waste canister 26, and the sprayers direct the liquid downwardly and radially outwardly (schematically represented by arrows) towards the inner surface 60. The flow surface 90 may be associated with the upper aspect 82 and slope downwardly and radially outwardly. In other words, the upper side 68 of the insert 58 may be thin, and the insert 58 tapers outwardly towards the imaging feature 74. FIG. 4 shows the lateral sides 68 associated with the upper aspect 82 being triangular in shape. Owing to the imaging feature 74 being spaced apart from the inner surface 60 of the waste canister 26, the flow surface 90 is also spaced apart from the inner surface 60, and increasingly so upwardly. Therefore, during operation of the cleaning subsystem 52, the pressurized liquid can reach an upper edge of the imaging feature 74 at least mostly unobstructed, maximizing its cleaning potential. The flow surface 90 may also provide for laminar flow of the pressurized liquid. The flow surface 90 may be provided at an angle, a, relative to horizontal within the range of approximately 30 to 80 degrees, and more particularly within the range of 40-50 degrees. It is contemplated that additional geometries may be associated with the flow surface 90, for example, channels, lumens, and undulations. Given the advantageous functionality of the flow surface 90, the thin layer of medical waste is better flushed from the gap between the imaging feature 74 and the inner surface 60, and the improved cleaning may limit staining or otherwise preserve the optical characteristics of the insert 58.

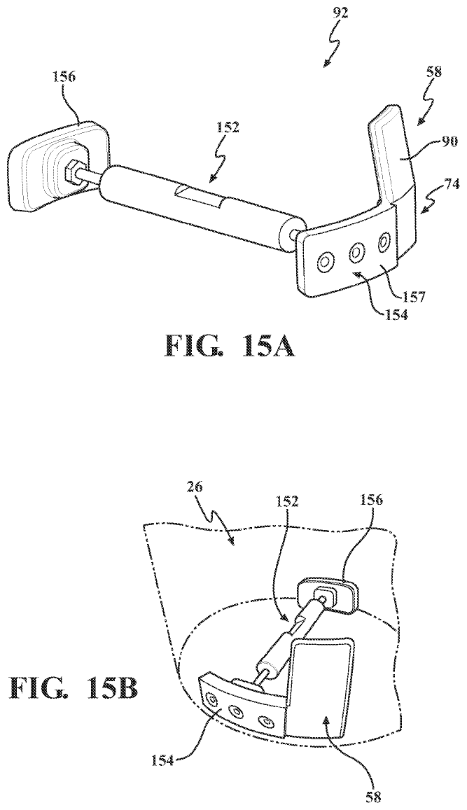

The first imaging surface 76 is shown to the right of the second imaging surface 78 (when viewed from the front side 64), but the reverse configuration is contemplated. The reverse configuration may be provided for image-based processing considerations. In certain implementations, the reverse configuration may be based on a direction of rotation of the sprayers of the cleaning subsystem 52. More particularly, the first and second imaging surfaces 76, 78 may be arranged so that the pressurized liquid contacts the first imaging surface 76 (i.e., closer to the inner surface 60) prior to contacting the second imaging surface 78. Should any semi-solid or solid debris be situated between the insert 58 and the inner surface 60, the aformentioned flow direction increases the likelihood of dislodging the debris (as opposed to urging it towards a face of the ridge 80). Further, the ridge 80 may be radiused or otherwise contoured, as mentioned, to improve cleanability regardless of the flow direction. It should be understood that the flow surface 90 may also be utilized on implementations in which the imaging feature 74 does not include the second imaging surface 78 (see FIG. 15A).

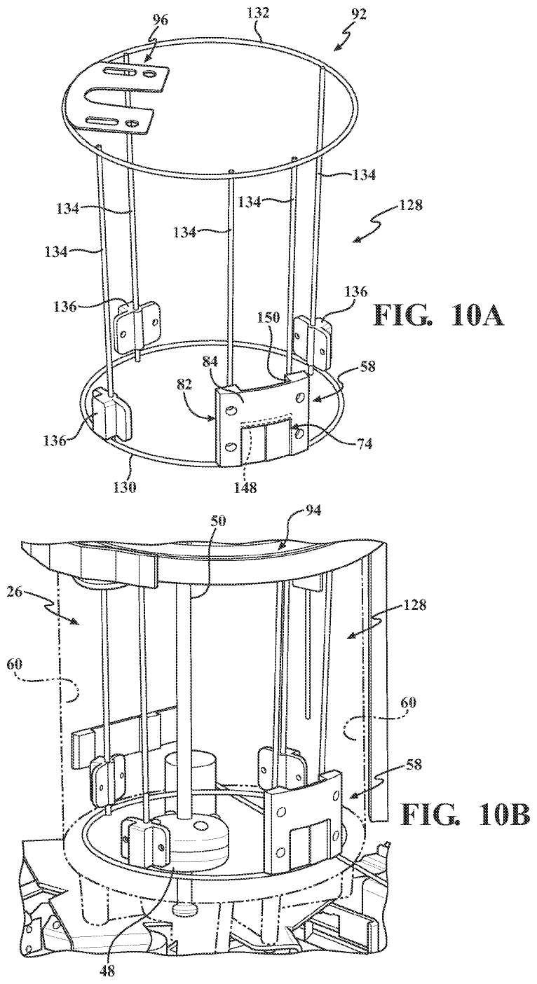

The insert 58 may be provided on an insert assembly 92. The insert assembly 92, in a broadest sense, includes the insert 58 and means for supporting the insert 58 within the waste canister 26 to locate and maintain a position the imaging feature 74. The means for supporting the insert 58 may couple the insert assembly 92 to the upper cap 94 of the waste canister 26 (see FIGS. 5A-6A and 7B), the lower portion 54 of the waste canister 26 (see FIGS. 9A and 9B), a sidewall of the waste canister 26 (see FIGS. 8A-8C and 15A-16B), or combinations thereof (see FIGS. 10A-15B).

The implementations of the insert assembly 92 of FIGS. 5A-7B include a mounting head 96, a strut 98, and the insert 58. The mounting head 96 configured to be coupled to the upper cap 94. The mounting head 96 may define at least one opening 100 configured to accommodate structures of the upper cap 94 such as the sensor rod 50 of the fluid measuring subsystem 42 and the sprayers of the cleaning subsystem 52. An end of the strut 98 is coupled to the mounting head 96, and another end of the strut 98 is coupled to the insert 58. In the implementation of FIGS. 5A and 5B, the strut 98 is a resiliently flexible plate 102. The plate 102 is coupled to the insert 58 near the upper side 68. The plate 102 is dimensioned and oriented relative to the mounting head 96 the insert 58 such that, with the mounting head 96 secured to the upper cap 94, the insert 58 is urged into direct contact with the inner surface 60. The plate 102 may be nearly perpendicular to the mounting head 96, and the insert 58 may be tilted slightly relative to the plate 102 so as to be vertically oriented for flush contact between the lower and upper aspects 70, 82 and the inner surface 60. In the implementation of FIGS. 6A and 7A, the strut 98 is a pair of resiliently flexible rods 104. The rods 104 are also dimensioned and oriented relative to the mounting head 96 the insert 58 such that, with the mounting head 96 secured to the upper cap 94, the insert 58 is urged into direct contact with the inner surface 60. The rods 104 are coupled to the lateral sides 68 of the insert 58 to provide a fulcrum. Owing to the differing construction, the rods 104 may provide less outward force on the insert 58 than that of the plate 102. The fulcrum permits the insert 58 to pivot to maintain optimal contact between the insert 58 and the inner surface 60. The insert 58 may be modified accordingly to further promote the optimal contact. For example, FIGS. 6A and 7A show the front side 64 of the insert 58 including feet 106. A depth of the feet 106 may dictate the gap between the imaging feature 74 and the inner surface 60. The feet 106 are in a triangular arrangement and sized relatively small to allow for wobble should all of the feet 106 not be in direct contact with the inner surface 60. The connection between the rods 104 and the insert 58 may also allow for wobble. The rods 104 urge the insert 58 towards the waste canister 26 with the wobble facilitating the insert 58 to “self-center” itself vertically against the inner surface 60. It is contemplated that the lesser outward force of the rods 104 in combination with the wobble may permit for brief separation of the insert 58 from the inner surface 60 from jostling by the pressurized liquid during operation of the cleaning subsystem 52, which may improve cleanability. It is noted that, relative to the plate 102, the rods 104 obstruct less of the pressurized liquid being directed radially outwardly from the sprinklers above. In particular, the rods 104 are spaced apart laterally to avoid obstructing the upper side 68 of the insert 58. Therefore, the flow surface 90 is well suited for the implementation of FIG. 7A. The strut 98 of the implementation of FIG. 5A may include apertures or slots (not identified) configured to provide fluid communication through the plate 102, in which instance the flow surface 90 may be provided.

Referring now to FIGS. 6A-6C, another implementation of the insert 58 and insert assembly 92 is shown in which the imaging feature 74 includes the first and second imaging surfaces 76, 78. The insert 58 is rectangular in shape with a width greater than a height. A thickness of the insert 58 defined between the front side 64 and the rear side 66 is relatively larger such that the insert 58 is block-like in construction. The increased thickness provides for greater rigidity of the insert 58 and sturdiness within the waste canister 26 against forces from turbulent fluids and the like. The insert 58 may define at least one cavity 164 extending forward from the rear side 66 of the insert 58. FIG. 6B shows two cavities 164 separated by a barrier 166. The cavities 164 minimize the volume of the insert 58 despite its relatively grater thickness. As mentioned, it is desirable to minimize the consumed subvolume so as to limit its effect on the fluid measuring subsystem 42. The rear side 66 may further define slots 168 through the opposing lateral sides 68 and, if present, the barrier 166 with the slots 168 sized to receive a portion the strut 98. The illustrated implementation shows the strut 98 in a U-shaped configuration with the rods 104 extending upwardly from a crossmember extending through the slots 168. FIG. 6A shows the first imaging surface 76 positioned to the left of the second imaging surface 78 (when viewed from the front side 64).

FIG. 6C shows a variant of the insert 58 of FIG. 6A in which the imaging feature 74 is formed of singular surface that tapers between the lateral sides 68 of the insert 58. In other words, the thickness of a first of the lateral sides 68 is greater than the thickness of a second of the lateral sides 68. The feet 106 may be correspondingly dimensioned to engage the inner surface 60 of the waste canister 26 to maintain the tapered profile of the imaging feature 74. With the feet 106 directly contacting the inner surface 60 of the waste canister 26, the tapered profile of the imaging feature 74 is calibration data known to the controller 44. For example, the tapered profile may include minimum and maximum distances of the gap between the insert 58 and the inner surface 60 may be approximately 1.7 millimeters and 2.2 millimeters, respectively. Further, the tapered profile of the imaging feature 74 may facilitate dislodging of debris and improved cleanability of the insert 58.

Referring now to FIGS. 8A-8C, the implementation of the insert assembly 92 includes a coupler housing 108, at least one magnet 110, and the insert 58. The magnets 110 are coupled to one of the coupler housing 108 and the insert 58 with the other one including ferromagnetic material. FIG. 7A generally shows the coupler housing 108 being formed with pockets sized to accommodate the magnets 110.

The coupler housing 108 is contoured to at least an outer surface of the waste canister 26. The coupler housing 108 may be further contoured to the lower portion 54 of the waste canister 26 to facilitate consistent positioning of the insert 58. More particularly, the coupler housing 108 may include lower member 112 that is flared and contoured to be flush with the lower portion 54 while an upper member 114 is flush with the outer surface. One of the magnets 110 may be disposed on the lower member 112, and another one of the magnets disposed on the upper member 114. The contour of the lower member 112 in combination with the magnet 110 may “self-align” the coupler housing 108 with a complementary contour of the lower portion 54. With the insert 58 being coupled to the coupler housing 108 at a fixed point (i.e., with the magnet 110), the consistent positioning of the insert 58 against the inner surface 60 of the waste canister 26 may be more easily performed during installation, service, and replacement. The consistent positioning may include a lower edge of the imaging feature 74 corresponding to a fluid volume of approximately 400 milliliters (as identified by volumetric markings on the waste canister 26 (see FIGS. 8C and 9B)). It is more broadly contemplated that the lower edge may correspond to a fluid volume within range of approximately 100 to 1000 milliliters, and more particularly within the range of 200 to 600 milliliters.

The consistent positioning may be further realized by aligning the coupler housing 108 with at least one rib 116 of the waste canister 26. As best shown in FIG. 8B, an underside of the lower portion 54 of the waste canister 26 includes the ribs 116. The ribs 116 may provide strength to the frustoconical-like shape of the lower portion 54. At least one of the ribs 116 may extend radially towards a front of the waste canister 26; i.e., a portion of the waste canister 26 to be visible through the window 38. Therefore, the rib 116 may be utilized to provide a rigid structure against which the coupler housing 108 may be situated to radially position the insert 58 within the waste canister 26 in a consistent manner. FIG. 8B shows an edge of the lower member 112 directly contacting the rib 116.

The illustrated implementation shows the lower member 112 and the upper member 114 in an L-shape arrangement. The upper member 114 is therefore positioned lateral to the insert 58, and the liquid from the cleaning subsystem 52 may reach the flow surface 90 of the insert 58 without obstruction. The insert 58 may include a dimple or post 118 to directly contact the inner surface 60 and further support the insert 58 in the desired position. Other geometries and arrangements of the coupler housing 108 and/or the insert 58 are contemplated to provide the aformentioned functionality with the magnets 110.

The insert 58 shown in FIGS. 9A and 9B is the same as that disclosed with reference to FIG. 4, and is hereby incorporated by reference. The insert assembly 92 is configured to secure the insert 58 to the sensor rod 50 of the fluid measuring subsystem 42. As generally appreciated from FIG. 9B, the sensor rod 50 is off-centered within the waste canister 26 and extends vertically from within the lower portion 54 to the upper cap 94. The insert assembly 92 includes a flange 120 extending from the insert 58 and coupled to a mounting hub 122. The flange 120 may extend from the lower side 68, the lateral side 68 or another portion of the insert 58. The mounting hub 122 defines an opening 123 sized greater than the sensor rod 50. The mounting hub 122 is coupled to the sensor rod 50 in the lower portion 54 of the waste canister 26, and the flange 120 is contoured to position the insert 58 adjacent to or in an abutting relationship with the inner surface 60.

A locking member 124 may secure the mounting hub 122 to the sensor rod 50. The locking member 124 defines an opening sized greater than the sensor rod 50, and fasteners configured to clamp the locking member 124 to the sensor rod 50. The locking member 124 and the mounting hub 122 include complementary orientation features 126 configured to engage one another such that, with the locking member 124 clamped to the sensor rod 50, the mounting hub 122 is prevented from rotating about the sensor rod 50. The insert 58 is correspondingly prevented from rotation and thereby locked in the desired position.

As previously mentioned, the lip 40 of the front casing 36 of the chassis 22 may obscure at least the lower portion 54 of the waste canister 26. In other words, the window 38 of the front casing 36 may not extend sufficiently downwardly for the lower portion 54 to be visualized. With the implementation of the insert assembly 92 of FIGS. 9A and 9B supported within the lower portion 54 of the waste canister 26, a height of the lower aspect 70 defined between the lower side 68 and the imaging feature 74 may be sized to position the imaging feature 74 above the lip 40 and within the window 38. It is understood that the lower aspect 70 is optional (see FIGS. 6A, 6B, 7A, 7B, 10A, 10B, 15A and 15B).

Referring to FIGS. 10A-13, implementations of the insert assembly 92 include a frame 128 that may be self-supporting and self-locating within the waste canister 26. Such implementations may be particularly well suited for retrofitting by requiring minimal or no modification to or assembly with the waste canister 26. The frame 128 of FIGS. 10A and 10B includes a lower support ring 130, an upper support ring 132, and braces 134 fixedly securing the upper support ring 132 to the lower support ring 130. The lower and upper support rings 130, 132 may be circular as shown, but other geometries are contemplated. The insert 58 is fixedly coupled to the frame 128, and optionally, stabilizers 136 may be coupled to the frame 128.

An outer diameter of the lower support ring 130 is sized to approximate an inner diameter of the waste canister 26 at a desired vertical position within the waste canister 26. In one example, the lower support ring 130 is sized to be situated on the frustoconical-like shape of the lower portion 54. Likewise, an inner diameter of the upper support ring 132 may be sized to approximate the inner diameter of the waste canister 26 at or near the upper cap 94 of the waste canister 26. Furthermore, the braces 134 may have a height defined between the lower support ring 130 and the upper support ring 132 to approximate a height of the waste canister 26. With insert assembly 92 disposed within the waste canister 26 and the upper cap 94 secured to the waste canister 26, the frame 128 is fixed in position. Lateral movement is prevented by the stabilizers 136 and vertical movement is prevented by the lower portion 54 and the upper cap 94. The insert assembly 92 may include the mounting head 96 secured to the frame 128 if is desired to mechanically couple the frame 128 to the upper cap 94. The insert 58 may be coupled to at least one of the braces 134, and/or to the lower support ring 130.

FIG. 11 shows a variant of the frame 128 in which the lower support ring 130 is a molded component and includes features 138 for improved coupling of the braces 134, and a front feature 140 for improved coupling of the insert 58. The front feature 140 may include a height sized to position the imaging feature 74 above the lip 40 and within the window 38. The front feature 140 may also include a mount 142 configured to secure the insert 58 to the frame 128, for example, with adhesive or another fastening means. The mount 142 may extend upwardly to a distance less than a height of the insert 58 such that the mount 142 is not visible through the window 38. Likewise, the front feature 140 including the mount 142 may be clocked about the lower support ring 130 by approximately ninety degrees from the braces 134 such that little to none of the insert assembly 92 is visible through the window 38 other than the insert 58.

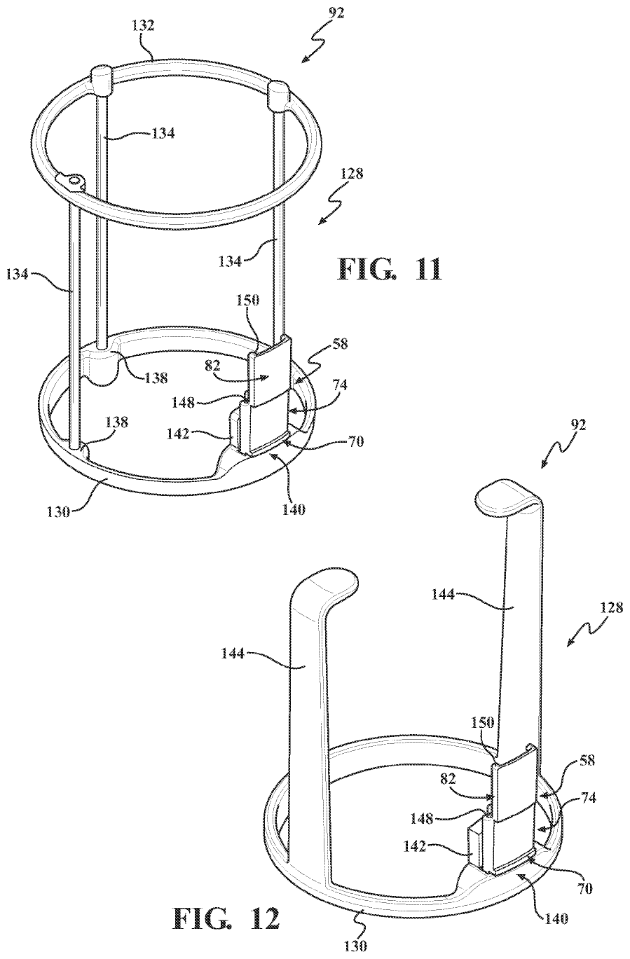

FIG. 12 shows another variant of the frame 128 in which resilient tongues 144 extend upwardly from the lower support ring 130. The tongues 144 are dimensioned and oriented relative to the lower support ring 130 to be resiliently deflected inwardly as the frame 128 is deposited into the waste canister 26. The frictional forces from the tongues 144 against the inner surface 60 of the waste canister 26 maintain the desired position of the insert 58.

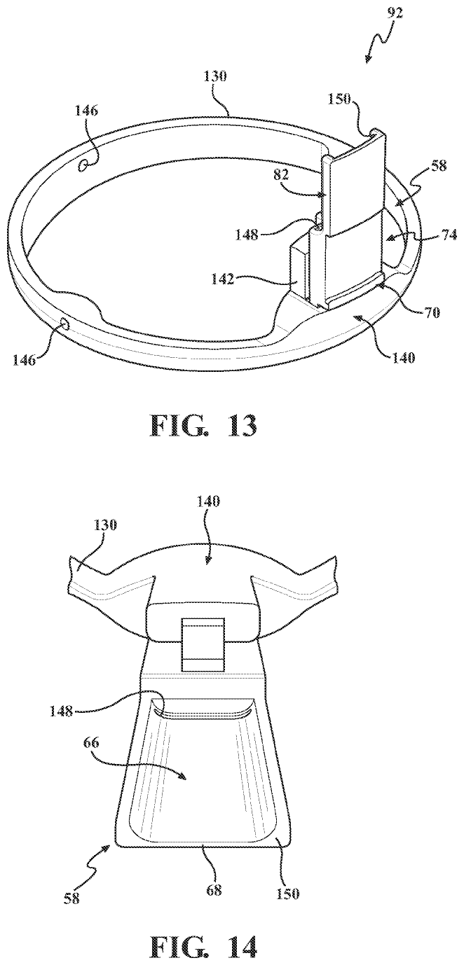

FIG. 13 shows still another variant of the frame 128 in which the lower support ring 130 defines apertures 146 configured to threadably receive set screws. The set screws may be placed in engagement with the inner surface 60 of the waste canister 26. The frame 128 does not include the upper support ring 132 or the braces 134, and therefore minimizes the consumed subvolume of the insert assembly 92 within the waste volume of the waste canister 26.

Referring again to FIGS. 10A-13 and with further reference to FIG. 14, it is observed that the implementations of the insert 58 do not include the flow surface 90, but rather include the front surface 84 contoured to (and configured to directly contact) the inner surface 60 of the waste canister 26. With the advantages of the flow surface 90 not realized, the pressurized liquid from the sprayers of the cleaning subsystem 52 may otherwise be prevented from cleaning the imaging feature 74 in an unobstructed manner. The insert 58 of these implementations may define a slot 148 providing fluid communication from the rear side 66 to the front side 64 of the insert 58. As best shown in FIG. 14, the slot 148 may be positioned widthwise along the insert 58 and located above the imaging feature 74. Further, the upper aspect 82 may define a channel 150 extending between the upper side 68 of the insert 58 and the slot 148. The channel 150 is in fluid communication with the slot 148 and configured to maximize an amount of the pressurized liquid encountering the rear side 66 to be directed through the slot 148. With the slot 148 being located above or adjacent the imaging feature 74, the pressurized liquid passing through the slot 148 contacts the imaging feature 74 with minimal loss in fluid velocity. The preserved fluid velocity may improve cleanability of the insert 58. It is understood that the insert 58 including the slot 148 may be provided on any of the implementations of the insert assembly 92 as described throughout the present disclosure.

FIGS. 15A and 15B show another implementation of the insert assembly 92 in which a turnbuckle 152 secures the insert 58 within the waste canister 26. The turnbuckle 152 movably couples a front mount 154 to a rear mount 156. The front mount 154 may include a front surface 157 contoured to the inner surface 60 of the waste canister 26. The imaging feature 74 may be positioned lateral to the front mount 154, but other suitable arrangements are contemplated. The flow surface 90 is positioned above the imaging feature 74. The turnbuckle 152 is operated in a known manner to urge separation of the front mount 154 and the rear mount 156 into engagement with counterpart aspects of the inner surface 60 of the waste canister 26.

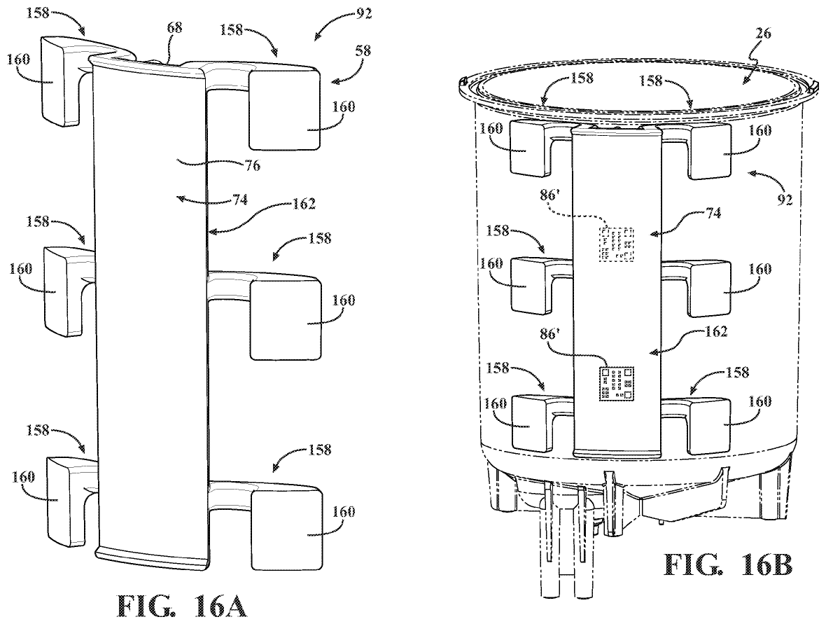

FIGS. 16A and 16B show another implementation of the insert assembly 92 in which adhesive is used to affix the insert 58 within the waste canister 26. The insert assembly 92 includes at least one leg 158 coupled to or integrally formed with the insert 58. The legs 158 are configured to be affixed to the waste canister 26 to position the first imaging surface 76 at the first distance from the inner surface 60. For example, each of the legs 158 may include a foot 160 including a front surface of sufficient surface area to support a weight of the insert 58 with adhesive. FIGS. 16A and 16B show the insert 58 including six legs 158 and six feet 160 with pairs of the legs 158 and the feet 160 extending in opposite directions from a body 162. More or less legs 158 may be provided, and the legs 158 may be arranged in any suitable configuration (e.g., asymmetric, angled, etc.).

The body 162 has a height defined between the upper and lower sides 68 that is sized to vertically traverse at least a majority of the height of the waste canister 26. The pairs of the legs 158 are spaced apart from each other vertically along the body 162. The illustrated implementation shows the pairs of the legs 158 equally spaced apart from another, but alternatively the pairs of the legs 158 may be weighted or stacked to an upper or lower portion of the body 162. The height of the body 162 provides for the imaging feature 74 that correspondingly vertically traverses at least a majority of the height of the waste canister 26. As such, the thin layer of fluid may be present at greater volumes of the waste material. The controller 44 may use a greater portion of the image from the optical sensor 88 for performing the image-based determination of the blood component to improve accuracy, and/or the controller 44 may perform multiple image-based determinations each corresponding to a predetermined region of the image along the body 162. At least one additional reference marker 86′ may be provided to facilitate locating the additional predetermined region of the imaging feature 74 to be analyzed. The multiple image-based determinations may be averaged or otherwise reconciled. Such arrangements may be particularly well suited in instances where the waste material is non-homogenous, for example, lighter-colored, less-dense irrigating fluids at least mildly separated from and situated atop the darker-colored, more-dense blood. Additionally or alternatively, it is contemplated that an impeller may be operable within the waste canister 26 to mix the waste material to improve homogeneity.

It is further contemplated that additional reference markers may be disposed on the insert 58, the waste canister 26, and/or other suitable location of the system to provide for light normalization in addition to locating the imaging feature 74. For example, three, four, or more QR codes (or other reference marker) may be affixed at radial positions about the waste canister 26 such that, regardless of a location of the mobile device 89 relative to the waste canister 26, the imaging feature 74 can be accurately determined and the ambient lighting can be accurately normalized. Another example include affixing the additional reference marker(s) to a top of the waste canister 26.

It should be appreciated the implementations of the insert 58 discussed herein and the means for supporting the insert 58 within the waste canister 26 are interchangeable. The insert 58 itself may be formed from a polymer, composite, or other suitable material. As mentioned, the material may be white, opaque, and impermeable to fluid within the waste canister 26. The insert 58 may be manufactured from a highly-reflective (i.e., glossy) material and/or coated with a glossy coating. Alternatively, the material may be non-white, non-opaque, and/or fluid permeable. The material and/or the coating may be designed to retard against staining for reusability of the insert assembly 92 for a period of time. For example, the cleanability and anti-staining features may require replacement every three, six, nine or more months. It is possible that the insert assembly 92 may not require replacement altogether.

Exemplary methods of using the insert assembly 92 to quantify the blood component includes selecting a first region of the image that corresponds to the layer of fluid between the first image surface 76 of the image feature 74 and the inner surface 60 along a line of sight from the optical sensor 88. Optionally, the controller 44 may select a second region of the image that corresponds to the layer of fluid between the second image surface 78 and the inner surface 60. The first region and the second region may be selected based on a location in the image of the reference marker 86.

The first and second distances provide the color gradient. Color parameters may be extracted from the first region and the second region. The color parameters are processed to determine the concentration of the blood component in the waste material. Exemplary implementations of the aforementioned U.S. Pat. No. 8,792,693 disclose using a parametric model or a template matching algorithm to determine the concentration of the blood component associated with fluid within the canister. A hemolysis status of the blood within the waste canister may be determined.

The controller 44 determines the fluid volume of the waste material based on data received from the fluid measuring subsystem 42, and further determines the blood loss based on the concentration of the blood component and the determined fluid volume. Since the fluid measuring subsystem 42 provides the data to the controller 44 in real time, the determined blood loss may be automatically updated upon capturing a new image with the optical sensor 88. It is further contemplated that the optical sensor 88 may be integrated onto the chassis 22 of the medical waste collection system 20 in which images of the waste canister 26 are captured continuously, or at regular intervals or selected instances during the medical procedure. The blood loss of the patient may be updated in real time on the user interface 46 without any involvement of the operator. Alternatively, the fluid volume may be manually entered on the user interface 46 or the mobile device 89 associated with the optical sensor 88. The mobile device 89 may be a tablet, smartphone, digital camera, or the like.

The methods may be computer-implemented by a machine configured to receive a non-transitory computer-readable medium storing computer-readable instructions. The computer-readable instructions may be executed by computer-executable components integrated with at least one of an application, applet, host, server, network, website, communication service, communication interface, hardware, firmware, software, or the like. The computer-readable medium can be stored on any suitable computer readable media such as RAM, ROM, flash memory, EEPROM, optical devices, hard drives, floppy drives, or the like. The computer-executable component may be a processor (of the controller 44 or a separate processor), but any suitable hardware device may execute the computer-readable instructions.

Several implementations have been discussed in the foregoing description. However, the implementations discussed herein are not intended to be exhaustive or limit the invention to any particular form. Modifications and variations are possible in light of the above teachings and may be practiced otherwise than as specifically described. For example, the blood component may be hemoglobin, or otherwise may be one or more of whole blood, red blood cells, platelets, plasma, white blood cells, analytes, and the like. The methods may also be used to estimate a concentration and an amount of a non-blood component within the waste canister 26, such as saline, ascites, bile, irrigating fluids, saliva, gastric fluid, mucus, pleural fluid, interstitial fluid, urine, fecal matter, or the like. The medical waste collection system 20 may communicate with other systems to form a fluid management ecosystem for generating a substantially comprehensive estimate of extracorporeal blood volume, total blood loss, patient euvolemia status, or the like.

Certain inventive aspects of the present disclosure are described with reference to the following exemplary clauses:

Clause 1—An insert assembly for quantifying a blood component in medical waste disposed within a waste canister of a medical waste collection system that further includes a vacuum source, and a cleaning subsystem including a rotatable sprayer for directing pressurized liquid towards an inner surface of the waste canister, the insert assembly comprising: an insert comprising a front side, a rear side, and an imaging feature comprising a first imaging surface configured to provide a first region of an image to be processed for quantifying the blood component; and means for positioning the insert within the waste canister to be adjacent to or abutting an inner surface of the waste canister, wherein the first imaging surface is configured to be spaced apart from the inner surface of the waste canister by a first distance, wherein the insert further comprises a flow surface configured to direct pressurized liquid from the cleaning subsystem towards the imaging feature, or defines a slot configured to permit the pressurized liquid to be directed from the cleaning subsystem towards the imaging feature.

Clause 2—The insert assembly of clause 1, wherein the slot provides fluid communication between the rear side and the front side of the insert.

Clause 3—The insert assembly of clause 2, wherein the slot is positioned adjacent and above the imaging feature.

Clause 4—The insert assembly of clause 1, wherein the flow surface is sloped and oriented to be aligned with an incoming direction of the pressurized liquid.

Clause 5—The insert assembly of any one of clauses 1-4, wherein the imaging feature further comprises a second imaging surface configured to be spaced apart from the inner surface of the waste canister by a second distance greater than the first distance, and wherein the first imaging surface is positioned laterally to the second imaging surface based on a rotational direction of sprayer of the cleaning subsystem so as to encounter the pressurized liquid prior to the second imaging surface.

Clause 6—An insert assembly for quantifying a blood component in medical waste disposed within a waste canister of a medical waste collection system that further includes a vacuum source, the insert assembly comprising: an insert comprising an imaging feature configured to be imaged with an optical sensor for the image be processed for quantifying the blood component; and means for positioning the insert within the waste canister to cause a front side of the insert to engage the inner surface of the waste canister, and to space apart the imaging feature from the inner surface.

Clause 7—The insert assembly of clause 6, wherein the means for positioning the insert within the waste canister comprises at least one of a flexible plate, a pair of flexible rods, a magnet, a locking member, a frame, tongues, a turnbuckle, and adhesive.

Clause 8—A method for quantifying blood loss in medical waste within a waste canister of a medical waste collection system that includes a vacuum source, a fluid measuring subsystem, an insert disposed within the waste canister, and a reference marker affixed to the waste canister, the method comprising: receiving an image of the waste canister; identifying a first region of the image based on the reference marker; determining a concentration of a blood component in waste material based on optical properties of the first region of the image; receiving data from the fluid measuring system indicative of a fluid level of the waste material in the waste canister; determining a fluid volume of the waste material; and quantifying the blood loss based on the determined concentration of the blood component and the determined fluid volume.

1. An insert assembly for quantifying a blood component in medical waste disposed within a waste canister of a medical waste collection system that includes a vacuum source, the insert assembly comprising:
an insert comprising an imaging feature configured to be imaged with an optical sensor for an image be processed for quantifying the blood component, wherein the imaging feature comprises a first imaging surface and a second imaging surface; and
means for positioning the insert within the waste canister to cause a front side of the insert to engage an inner surface of the waste canister, space apart the first imaging surface from the inner surface by a first distance, and space apart the second imaging surface from the inner surface by a second distance greater than the first distance,
wherein the first imaging surface and the second imaging surface are positioned lateral to one another in a side-by-side arrangement.

2. The insert assembly of claim 1, wherein the imaging feature is rectangular, and wherein each of the first imaging surface and the second imaging surface are square or rectangular.

3. The insert assembly of claim 1, wherein the first distance is within a range of 1.2 to 3.7 millimeters, and wherein the second distance is within a range of 1.7 to 4.2 millimeters.

4. The insert assembly of claim 1, wherein the first imaging surface and the second imaging surface are separated by a ridge having a thickness equal to a difference between the first distance and the second distance.

5. (canceled)

6. The insert assembly of claim 1, wherein the insert further comprises at least three feet extending forward of the imaging feature and configured to directly contact the inner surface of the waste canister.

7. The insert assembly of claim 6, wherein the at least three feet are in a triangular arrangement so as to allow for wobble and vertical self-centering of the insert against the inner surface of the waste canister.

8. (canceled)

9. The insert assembly of claim 1, wherein the means for positioning the insert within the waste canister comprises a mounting head configured to be coupled to an upper cap of the waste canister, and a strut coupling the mounting head with the insert, wherein the strut is biased or formed to urge the insert into direct contact with the inner surface of the waste canister.

10. The insert assembly of claim 9, wherein the insert defines at least one slot within which the strut is pivotably disposed so as to permit the insert to pivot about the strut.

11. The insert assembly of claim 9, wherein the strut further comprises rods coupled to one another with a crossmember in a U-shaped arrangement.

12. The insert assembly of claim 1, wherein the means for positioning the insert within the waste canister comprises at least one of a magnet, a locking member, a frame, tongues, a turnbuckle, and adhesive.

13. The insert assembly of claim 1, wherein the insert further comprises a lower aspect defined between a lower side of the insert and the imaging feature, wherein the lower aspect is sized relative to a lip of the medical waste collection system to position the imaging feature within a window of the medical waste collection system through which the waste canister is visible.

14. The insert assembly of claim 1, wherein the insert further comprises an upper aspect defined between an upper side of the insert and the imaging feature, wherein the imaging feature is recessed from the upper aspect.

15. The insert assembly of claim 1, wherein the medical waste collection system further includes a cleaning subsystem including a rotatable sprayer for directing pressurized liquid towards an inner surface of the waste canister, and wherein the first imaging surface is positioned relative to the second imaging surface based on a direction of the rotatable sprayer so as to encounter the pressurized liquid prior to the second imaging surface.

16. The insert assembly of claim 15, wherein the insert further comprises a flow surface configured to direct the pressurized liquid from the cleaning subsystem towards the imaging feature, and wherein the flow surface is sloped and oriented to be aligned with an incoming direction of the pressurized liquid.

17-19. (canceled)

20. An insert assembly for quantifying a blood component in medical waste disposed within a waste canister of a medical waste collection system that further includes a vacuum source, the insert assembly comprising:
a mounting head configured to be coupled to an upper cap of the waste canister;
a strut coupled to the mounting head; and
an insert coupled to the strut and comprising an imaging feature configured to be imaged with an optical sensor for an image be processed for quantifying the blood component,
wherein the strut is biased or formed to urge the insert into direct contact with an inner surface of the waste canister for the imaging feature to be spaced apart from the inner surface.

21. The insert assembly of claim 20, wherein the insert defines at least one slot within which the strut is pivotably disposed and configured to permit the insert to pivot about the strut.

22. An insert assembly for quantifying a blood component in medical waste disposed within a waste canister of a medical waste collection system that further includes a vacuum source, the insert assembly comprising:
an insert comprising an imaging feature configured to be imaged with an optical sensor for an image be processed for quantifying the blood component, wherein the imaging feature has a tapered profile extending between opposing lateral sides of the insert; and
means for positioning the insert within the waste canister to cause a front side of the insert to engage an inner surface of the waste canister, and to space apart the imaging feature from the inner surface.

23. The insert assembly of claim 22, wherein the means for positioning the imaging feature comprises feet extending from the front side of the insert and configured to directly contact the inner surface of the waste canister, wherein the feet are of differing lengths based on the tapered profile of the imaging feature.

24. The insert assembly of claim 23, wherein the feet are in a triangular arrangement so as to allow for wobble and vertical self-centering of the insert against the inner surface of the waste canister.

25. The insert assembly of claim 22, wherein the means for positioning the imaging feature comprises an upper aspect defined between an upper side of the insert and the imaging feature, wherein the upper aspect is contoured to and configured to engage the inner surface of the waste canister, and wherein the imaging feature is recessed from the upper aspect.
